(19) United States
(12) Patent Application Publication
(10) Pub. No.: US 2022/0259177 A1
(43) Pub. Date: Aug. 18, 2022

(54) METHODS FOR THE PREPARATION OF 5-BROMO-2-(3-CHLORO-PYRIDIN-2-YL)-2H-PYRAZOLE-3-CARBOXYLIC ACID

(71) Applicants: FMC Corporation, Philadelphia, PA (US); FMC Agro Singapore Pte. Ltd., Singapore (SG)

(72) Inventors: Yanchun CAO, Shanghai (CN); Xin LIU, Shanghai (CN); Jianhua MAO, Shanghai (CN); Zhijian XU, Shanghai (CN)

(21) Appl. No.: 17/769,783

(22) PCT Filed: Oct. 16, 2020
(86) PCT No.: PCT/US20/55885

§ 371 Date: Apr. 18, 2022

Related U.S. Application Data
(60) Provisional application No. 62/916,836, filed on Oct. 18, 2019.
(60) Provisional application No. 62/931,320, filed on Nov. 6, 2019.

(51) Int. Cl. C07D 401/04; C07D 231/16

(52) U.S. Cl. CPC C07D 401/04; C07D 231/16

(57) ABSTRACT

Described herein are novel methods of synthesizing 5-Bromo-2-(3-chloro-pyridin-2-yl)-2H-pyrazole-3-carboxylic acid from pyrazole or pyrazole derivatives. Also described herein are novel reaction intermediates.

### CROSS-REFERENCE TO RELATED APPLICATION

This application claims the benefit of U.S. Provisional Application No. 62/916,836 filed Oct. 18, 2019 and of U.S. Provisional Application No. 62/931,320 filed Nov. 6, 2019.

### FIELD OF INVENTION

This disclosure is directed to novel methods of synthesizing 5-Bromo-2-(3-chloro-pyridin-2-yl)-2H-pyrazole-3-carboxylic acid. Compounds prepared by the methods disclosed herein are useful for preparation of certain anthranilamide compounds that are of interest as insecticides, such as, for example, the insecticides chlorantraniliprole and cyantraniliprole.

### BACKGROUND

Conventional processes for the production of 5-Bromo-2-(3-chloro-pyridin-2-yl)-2H-pyrazole-3-carboxylic acid are subject to several industrial concerns, such as processability, environmental hazards, high cost, reagent reactivity, and necessary specialized equipment.

The present disclosure provides novel methods useful for preparing 5-Bromo-2-(3-chloro-pyridin-2-yl)-2H-pyrazole-3-carboxylic acid and derivatives thereof. The benefits of the methods of the present disclosure compared to previous methods are numerous and include improved overall yield, reduced cost, eliminated need for mixed solvent separations, reduced waste, simplified operation complexity, and reduced process hazards.

The disclosed methods provide an overall yield of about 50% with commercially available and easily handled reagents.

### BRIEF DESCRIPTION

In one aspect, provided herein is a method of preparing a compound of Formula VI, wherein

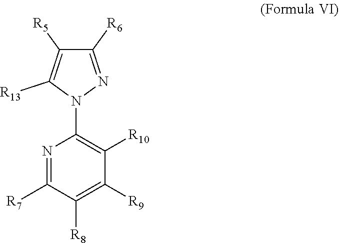

each of R₅-R₁₀ is independently selected from hydrogen and halogen; and
R₁₃ is an organic acid, the method comprising

I) forming a mixture comprising
A) a compound of Formula III, wherein

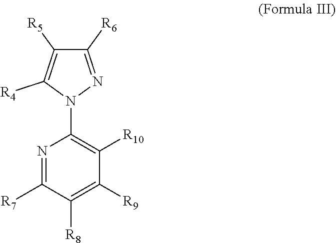

R4 is hydrogen; and
each of R₅-R₁₀ is independently selected from hydrogen and halogen;
B) a carbonyl-containing compound;
C) a solvent;
D) a compound comprising a metal; and
E) optionally an additive; and

II) reacting the mixture.

In one aspect, provided herein is a method of preparing a compound of Formula III, wherein

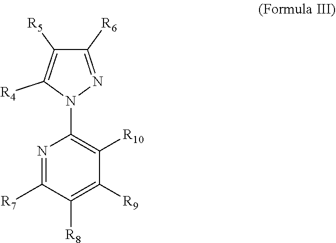

R₄ is hydrogen; and
each of R₅-R₁₀ is independently selected from hydrogen and halogen, the method comprising

I) forming a mixture comprising
A) a compound of Formula II, wherein

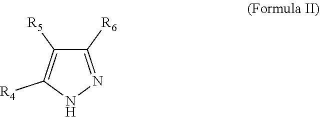

each of R₄, R₅, and R₆ is independently selected from hydrogen and halogen;
wherein at least one of R₄, R₅, and R₆ is hydrogen; and
wherein the compound of Formula II is prepared according to a method comprising
   i) forming a mixture comprising
      a) one or more compounds of Formula I, wherein

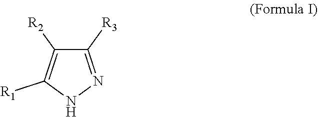

each of R₁, R₂, and R₃ is independently selected from halogen and hydrogen; and
wherein at least two of R₁, R₂, and R₃ are halogen;
   b) optionally a dehalogenation reagent;
   c) a reducing agent; and
   d) a solvent; and
   ii) reacting the mixture.
B) a compound of Formula IV, wherein

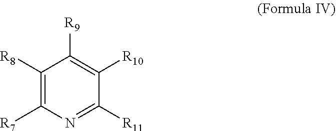

each of R₇-R₁₁ is independently selected from hydrogen and halogen;
C) a solvent;
D) an inorganic base; and
E) optionally an additive; and

II) reacting the mixture.

In one aspect, provided herein is a method of preparing a compound of Formula II, wherein

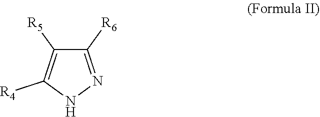

each of R₄, R₅, and R₆ is independently selected from hydrogen and halogen; and
wherein at least one of R₄, R₅, and R₆ is hydrogen, the method comprising

I) forming a mixture comprising
A) one or more compounds of Formula I, wherein

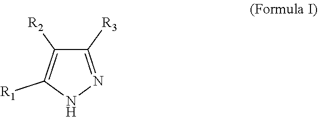

each of R₁, R₂, and R₃ is independently selected from halogen and hydrogen;
wherein at least two of R₁, R₂, and R₃ are halogen; and
wherein the one or more compounds of Formula I are prepared according to a method comprising
   i) forming a mixture comprising
      a) pyrazole or a pyrazole derivative;
      b) a halogenation reagent;
      c) water; and
      d) optionally an inorganic base; and
   ii) reacting the mixture.
B) optionally a dehalogenation reagent;
C) a reducing agent; and
D) a solvent; and

II) reacting the mixture.

In one aspect, provided herein is a method of preparing one or more compounds of Formula I, wherein

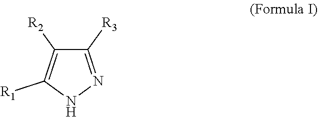

each of R₁, R₂, and R₃ is independently selected from halogen and hydrogen; and
wherein at least two of R₁, R₂, and R₃ are halogen, the method comprising

I) forming a mixture comprising
A) pyrazole or a pyrazole derivative;
B) a halogenation reagent;
C) water; and
D) optionally an inorganic base; and

II) reacting the mixture.

In one aspect, provided herein is a method of preparing a compound of Formula VI, wherein

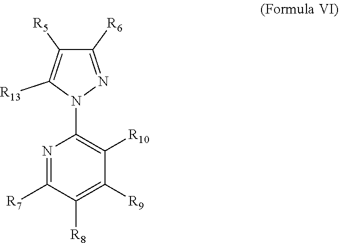

each of R₅-R₁₀ is independently selected from hydrogen and halogen; and
R₁₃ is an organic acid, the method comprising

I) forming a mixture comprising
A) a compound of Formula V, wherein

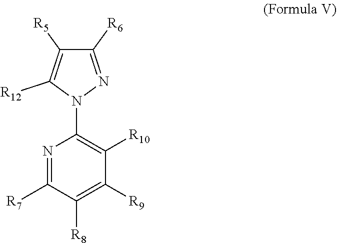

each of R₅-R₁₀ is independently selected from hydrogen and halogen;
R₁₂ is selected from ether, ester, and nitrile; and
wherein the compound of Formula V is prepared according to a method comprising
   i) forming a mixture comprising
      a) a compound of Formula III, wherein

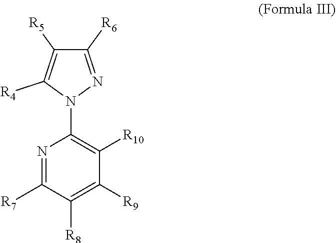

R₄ is hydrogen; and
each of R₅-R₁₀ is independently selected from hydrogen and halogen;
b) a solvent;
c) an organic compound;
d) a compound comprising a metal; and
e) optionally an additive; and
ii) reacting the mixture; and
B) a metal hydroxide; and

II) reacting the mixture; and

III) adding an acid to the mixture.

In one aspect, provided herein is a method of preparing a compound of Formula V, wherein

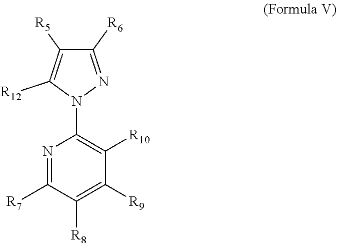

each of R₅-R₁₀ is independently selected from hydrogen and halogen;
R₁₂ is selected from ether, ester, and nitrile, the method comprising

I) forming a mixture comprising
A) a compound of Formula III, wherein

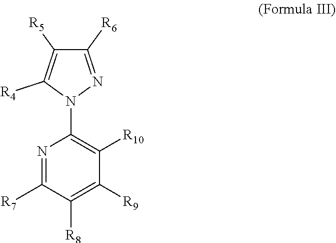

R₄ is hydrogen; and
each of R₅-R₁₀ is independently selected from hydrogen and halogen;
B) a solvent;
C) an organic compound;
D) a compound comprising a metal; and
E) optionally an additive; and

II) reacting the mixture.

In one aspect, provided herein is a method of preparing a compound of Formula II-A, wherein

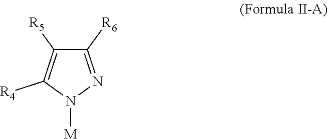

M is selected from alkali metals and alkaline metals;
each of R₄, R₅, and R₆ is independently selected from hydrogen and halogen; and
wherein at least one of R₄, R₅, and R₆ is hydrogen, the method comprising

I) forming a mixture comprising
A) one or more compounds of Formula I, wherein

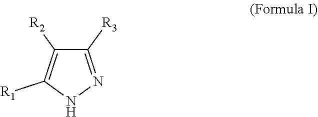

each of R₁, R₂, and R₃ is independently selected from halogen and hydrogen;
wherein at least two of R₁, R₂, and R₃ are halogen; and
wherein the one or more compounds of Formula I are prepared according to a method comprising
   i) forming a mixture comprising
      a) pyrazole or a pyrazole derivative;
      b) a halogenation reagent;
      c) water; and
      d) optionally an inorganic base; and
   ii) reacting the mixture.
B) optionally a dehalogenation reagent;
C) a reducing agent; and
D) a solvent; and

II) reacting the mixture.

In one aspect, provided herein is a compound of Formula II-A, wherein

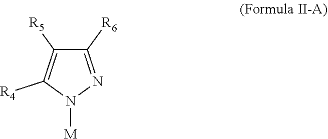

M is selected from alkali metals and alkaline metals;
each of R₄, R₅, and R₆ is independently selected from hydrogen and halogen; and
wherein at least one of R₄, R₅, and R₆ is hydrogen.

### DETAILED DESCRIPTION OF THE DISCLOSURE

As used herein, the terms “comprises,” “comprising,” “includes,” “including,” “has,” “having,” “contains”, “containing,” “characterized by” or any other variation thereof, are intended to cover a non-exclusive inclusion, subject to any limitation explicitly indicated. For example, a composition, mixture, process or method that comprises a list of elements is not necessarily limited to only those elements but may include other elements not expressly listed or inherent to such composition, mixture, process or method.

The transitional phrase “consisting of” excludes any element, step, or ingredient not specified. If in the claim, such would close the claim to the inclusion of materials other than those recited except for impurities ordinarily associated therewith. When the phrase “consisting of” appears in a clause of the body of a claim, rather than immediately following the preamble, it limits only the element set forth in that clause; other elements are not excluded from the claim as a whole.

The transitional phrase “consisting essentially of” is used to define a composition or method that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”.

Where an invention or a portion thereof is defined with an open-ended term such as “comprising,” it should be readily understood that (unless otherwise stated) the description should be interpreted to also describe such an invention using the terms “consisting essentially of” or “consisting of.”

Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Also, the indefinite articles “a” and “an” preceding an element or component of the invention are intended to be nonrestrictive regarding the number of instances (i.e. occurrences) of the element or component. Therefore “a” or “an” should be read to include one or at least one, and the singular word form of the element or component also includes the plural unless the number is obviously meant to be singular.

As used herein, the term “about” means plus or minus 10% of the value.

The term “halogen”, either alone or in compound words such as “haloalkyl”, includes fluorine, chlorine, bromine or iodine. Further, when used in compound words such as “haloalkyl”, said alkyl may be partially or fully substituted with halogen atoms which may be the same or different.

When a group contains a substituent which can be hydrogen, for example R⁴, then, when this substituent is taken as hydrogen, it is recognized that this is equivalent to said group being unsubstituted.

The term “organic base” includes, without limitation, amine compounds (e.g., primary, secondary and tertiary amines), heterocycles including nitrogen-containing heterocycles, and ammonium hydroxide.

The term “inorganic base” includes, without limitation, inorganic compounds with the ability to react with, or neutralize, acids to form salts, such as, for example, metal salts of hydroxide, carbonate, bicarbonate and phosphate.

The term “halogenation reagent” includes, without limitation, halogens and inorganic compounds, such as, for example, bromine, NBS, and 1,3-dibromo-5,5-dimethylhylhydantoin.

The term “phase transfer catalyst” includes compounds that facilitate the migration of a reactant from one phase into another phase where a reaction occurs. Phase transfer catalysis refers to the acceleration of the reaction upon the addition of the phase transfer catalyst.

The term “ester” includes, without limitation, a functional group comprising an ester bond (C(═O)—O—). In some aspects, the functional group comprising an ester bond is an alkyl (or cycloalkyl) having one to eight carbon atoms, like methyl, ethyl, 1-propyl, 2-propyl, 1-butyl, 1-methylheptyl (meptyl), etc.

The term “ether” includes, without limitation, a functional group comprising an ether bond (C—O—C).

The term “nitrile” includes, without limitation, a functional group comprising a nitrile bond (—C≡N).

The term “carboxylic acid” includes, without limitation, a functional group comprising a carboxylic acid bond (C(═O)—OH).

The term “organic acid” includes, without limitation, a functional group that confers acidity and consists of atoms selected from carbon, nitrogen, oxygen, and hydrogen.

Certain compounds of this invention can exist as one or more stereoisomers. The various stereoisomers include enantiomers, diastereomers, atropisomers and geometric isomers. One skilled in the art will appreciate that one stereoisomer may be more active and/or may exhibit beneficial effects when enriched relative to the other stereoisomer(s) or when separated from the other stereoisomer(s). Additionally, the skilled artisan knows how to separate, enrich, and/or to selectively prepare said stereoisomers.

The embodiments of this disclosure include:

Embodiment 1. A method of preparing a compound of Formula VI, wherein

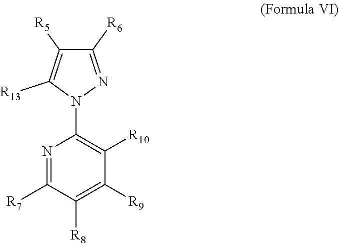

each of R₅-R₁₀ is independently selected from hydrogen and halogen; and

R₁₃ is an organic acid, the method comprising

I) forming a mixture comprising
A) a compound of Formula III, wherein

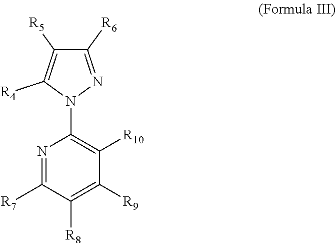

R₄ is hydrogen; and
each of R₅-R₁₀ is independently selected from hydrogen and halogen;
B) a carbonyl-containing compound;
C) a solvent;
D) a compound comprising a metal; and
E) optionally an additive; and

II) reacting the mixture.

Embodiment 2. The method of embodiment 1, wherein the compound comprising a metal is selected from a Grignard reagent and a lithium-containing compound.

Embodiment 3. The method of embodiment 2, wherein the Grignard reagent is selected from MeMgCl, iPrMgCl, iPrMgBr, EtMgCl, iPr₂NMgCl, iPr₂NMgBr, Et₂NMgCl, TMPMgCl, TMPMgCl.LiCl, iPr₂NMgCl.LiCl, iPr₂NMgBr.LiCl, and combinations thereof.

Embodiment 4. The method of embodiment 3, wherein the Grignard reagent is iPr₂NMgCl.

Embodiment 5. The method of embodiment 2, wherein the lithium-containing compound is selected from LDA, nBuLi, and combinations thereof.

Embodiment 6. The method of embodiment 1, wherein the solvent is selected from THF, toluene, 1,4-dioxane, Me-THF, and combinations thereof.

Embodiment 7. The method of embodiment 6, wherein the solvent is THF.

Embodiment 8. The method of embodiment 1, wherein the carbonyl-containing compound is selected from dimethylcarbonate, N,N-dimethylacetamide, carbon dioxide, and combinations thereof.

Embodiment 9. The method of embodiment 8, wherein the carbonyl-containing compound is carbon dioxide.

Embodiment 10. The method of embodiment 1, wherein the method step of reacting the mixture occurs at a reaction temperature in the range of about 0° C. to about 60° C.

Embodiment 11. The method of embodiment 10, wherein the method step of reacting the mixture occurs at a reaction temperature in the range of about 0° C. to about 30° C.

Embodiment 12. The method of embodiment 1, wherein R₅ and R₆ of Formula III are each independently hydrogen.

Embodiment 13. The method of embodiment 1, wherein the compound of Formula III, is prepared according to a method comprising

I) forming a mixture comprising
A) a compound of Formula II, wherein

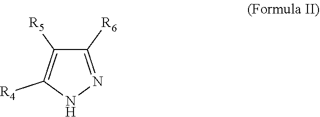

each of R₄, R₅, and R₆ is independently selected from hydrogen and

halogen; and
wherein at least one of R₄, R₅, and R₆ is hydrogen;
B) a compound of Formula IV, wherein

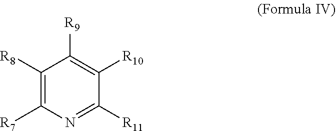

each of R₇-R₁₁ is independently selected from hydrogen and halogen;
C) a solvent;
D) an inorganic base; and
E) optionally an additive; and

II) reacting the mixture.

Embodiment 14. The method of embodiment 13, wherein the inorganic base is selected from powder sodium hydroxide, powder potassium hydroxide, potassium carbonate, potassium phosphate, powder sodium methoxide, powder potassium t-butoxide, and combinations thereof.

Embodiment 15. The method of embodiment 13, wherein the solvent is selected from toluene, N,N-dimethylformamide, N,N-dimethylacetamide, N-methyl-2-pyrrolidone, acetonitrile, and combinations thereof.

Embodiment 16. The method of embodiment 13, wherein the additive is selected from potassium iodide, a phase transfer catalyst, and combinations thereof.

Embodiment 17. The method of embodiment 16, wherein the phase transfer catalyst is selected from butyl ammonium chloride, tetra butyl ammonium bromide, aliquat-336, 18-crown-6, and combinations thereof.

Embodiment 18. The method of embodiment 13, wherein the method step of reacting the mixture occurs at a reaction temperature in the range of about 100° C. to about 200° C.

Embodiment 19. The method of embodiment 13, wherein the compound of Formula II is prepared according to a method comprising

I) forming a mixture comprising
A) one or more compounds of Formula I, wherein

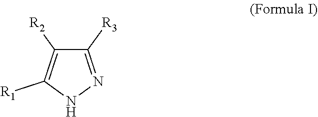

each of R₁, R₂, and R₃ is independently selected from halogen and hydrogen; and
wherein at least two of R₁, R₂, and R₃ are halogen;
B) optionally a dehalogenation reagent;
C) a reducing agent; and
D) a solvent; and

II) reacting the mixture.

Embodiment 20. The method of embodiment 19, wherein the solvent is selected from acetic acid, water, toluene, N,N-dimethylformamide, N,N-dimethylacetamide, and combinations thereof.

Embodiment 21. The method of embodiment 19, wherein the reducing agent is selected from sodium sulfite, sodium bisulfite, sodium hyposulfite, sodium thiosulfate, sodium hydrosulfide, sodium sulfate, and combinations thereof.

Embodiment 22. The method of embodiment 19, wherein the dehalogenation reagent is selected from sodium iodide, iodine, potassium iodide, tetra-n-butyl ammonium iodide, and combinations thereof.

Embodiment 23. The method of embodiment 19, wherein the method step of reacting the mixture occurs at a reaction temperature in the range of about 100° C. to about 180° C.

Embodiment 24. The method of embodiment 19, wherein the one or more compounds of Formula I are prepared according to a method comprising

I) forming a mixture comprising
A) pyrazole or a pyrazole derivative;
B) a halogenation reagent;
C) water; and
D) optionally an inorganic base; and

II) reacting the mixture.

Embodiment 25. The method of embodiment 24, wherein the halogenation reagent comprises

A) a reagent selected from hydrogen bromide, bromine, N-bromosuccinimide, 1,3-dibromo-5,5-dimethylhylhydantoin, sodium bromide, potassium bromide, and combinations thereof; and

B) optionally hydrogen peroxide.

Embodiment 26. The method of embodiment 24, wherein the inorganic base is selected from powder sodium hydroxide, sodium hydroxide solution, powder sodium acetate, and combinations thereof.

Embodiment 27. The method of embodiment 24, wherein the method step of reacting the mixture occurs at a reaction temperature in the range of about 0° C. to about 70° C.

Embodiment 28. The method of embodiment 24, wherein the halogenation reagent is a mixture of about 1 part by weight hydrogen peroxide and about 2 parts by weight hydrogen bromide.

Embodiment 29. A method of preparing a compound of Formula III, wherein

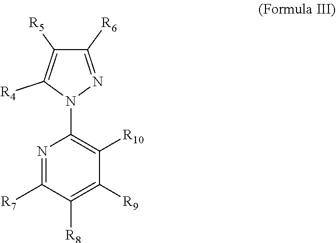

R₄ is hydrogen; and

each of R₅-R₁₀ is independently selected from hydrogen and halogen, the method comprising

I) forming a mixture comprising
A) a compound of Formula II, wherein

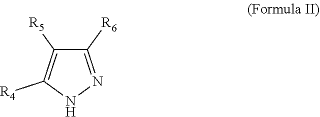

each of R₄, R₅, and R₆ is independently selected from hydrogen and halogen;
wherein at least one of R₄, R₅, and R₆ is hydrogen; and
wherein the compound of Formula II is prepared according to a method comprising
   i) forming a mixture comprising
      a) one or more compounds of Formula I, wherein

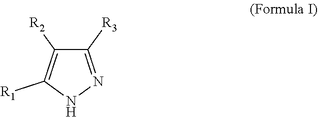

each of R₁, R₂, and R₃ is independently selected from halogen and hydrogen; and
wherein at least two of R₁, R₂, and R₃ are halogen;
b) optionally a dehalogenation reagent;
c) a reducing agent; and
d) a solvent; and

ii) reacting the mixture.
B) a compound of Formula IV, wherein

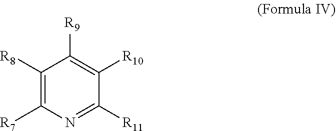

each of R₇-R₁₁ is independently selected from hydrogen and

halogen;
C) a solvent;
D) an inorganic base; and
E) optionally an additive; and

II) reacting the mixture.

Embodiment 30. The method of embodiment 29, wherein the inorganic base is selected from powder sodium hydroxide, powder potassium hydroxide, potassium carbonate, potassium phosphate, powder sodium methoxide, powder potassium t-butoxide, and combinations thereof.

Embodiment 31. The method of embodiment 29, wherein the solvent C) is selected from toluene, N,N-dimethylformamide, N,N-dimethylacetamide, N-methyl-2-pyrrolidone, acetonitrile, and combinations thereof.

Embodiment 32. The method of embodiment 29, wherein the additive is selected from potassium iodide, a phase transfer catalyst, and combinations thereof.

Embodiment 33. The method of embodiment 32, wherein the phase transfer catalyst is selected from butyl ammonium chloride, tetra butyl ammonium bromide, aliquat-336, 18-crown-6, and combinations thereof.

Embodiment 34. The method of embodiment 29, wherein the method step II) of reacting the mixture occurs at a reaction temperature in the range of about 100° C. to about 200° C.

Embodiment 35. The method of embodiment 29, wherein the solvent d) is selected from acetic acid, water, toluene, N,N-dimethylformamide, N,N-dimethylacetamide, and combinations thereof.

Embodiment 36. The method of embodiment 29, wherein the reducing agent is selected from sodium sulfite, sodium bisulfite, sodium hyposulfite, sodium thiosulfate, sodium hydrosulfide, sodium sulfate, and combinations thereof.

Embodiment 37. The method of embodiment 29, wherein the dehalogenation reagent is selected from sodium iodide, iodine, potassium iodide, tetra-n-butyl ammonium iodide, and combinations thereof.

Embodiment 38. The method of embodiment 29, wherein the method step ii) of reacting the mixture occurs at a reaction temperature in the range of about 100° C. to about 180° C.

Embodiment 39. The method of embodiment 29, wherein the one or more compounds of Formula I are prepared according to a method comprising

I) forming a mixture comprising
A) pyrazole or a pyrazole derivative;
B) a halogenation reagent;
C) water; and
D) optionally an inorganic base; and

II) reacting the mixture.

Embodiment 40. The method of embodiment 39, wherein the halogenation reagent comprises

A) a reagent selected from hydrogen bromide, bromine, N-bromosuccinimide, 1,3-dibromo-5,5-dimethylhylhydantoin, sodium bromide, potassium bromide, and combinations thereof; and

B) optionally hydrogen peroxide.

Embodiment 41. The method of embodiment 39, wherein the inorganic base is selected from powder sodium hydroxide, sodium hydroxide solution, powder sodium acetate, and combinations thereof.

Embodiment 42. The method of embodiment 39, wherein the method step of reacting the mixture occurs at a reaction temperature in the range of about 0° C. to about 70° C.

Embodiment 43. The method of embodiment 39, wherein the halogenation reagent is a mixture of about 1 part by weight hydrogen peroxide and about 2 parts by weight hydrogen bromide.

Embodiment 44. A method of preparing a compound of Formula II, wherein

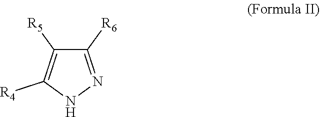

each of R₄, R₅, and R₆ is independently selected from hydrogen and halogen; and
wherein at least one of R₄, R₅, and R₆ is hydrogen, the method comprising

I) forming a mixture comprising
A) one or more compounds of Formula I, wherein

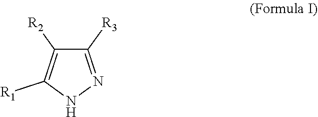

each of R₁, R₂, and R₃ is independently selected from halogen and hydrogen;
wherein at least two of R₁, R₂, and R₃ are halogen; and
wherein the one or more compounds of Formula I are prepared according to a method comprising
   i) forming a mixture comprising
      a) pyrazole or a pyrazole derivative;
      b) a halogenation reagent;
      c) water; and
      d) optionally an inorganic base; and
   ii) reacting the mixture.
B) optionally a dehalogenation reagent;
C) a reducing agent; and
D) a solvent; and

II) reacting the mixture.

Embodiment 45. The method of embodiment 44, wherein the solvent is selected from acetic acid, water, toluene, N,N-dimethylformamide, N,N-dimethylacetamide, and combinations thereof.

Embodiment 46. The method of embodiment 44, wherein the reducing agent is selected from sodium sulfite, sodium bisulfite, sodium hyposulfite, sodium thiosulfate, sodium hydrosulfide, sodium sulfate, and combinations thereof.

Embodiment 47. The method of embodiment 44, wherein the dehalogenation reagent is selected from sodium iodide, iodine, potassium iodide, tetra-n-butyl ammonium iodide, and combinations thereof.

Embodiment 48. The method of embodiment 44, wherein the method step II) of reacting the mixture occurs at a reaction temperature in the range of about 100° C. to about 180° C.

Embodiment 49. The method of embodiment 44, wherein the halogenation reagent comprises

A) a reagent selected from hydrogen bromide, bromine, N-bromosuccinimide, 1,3-dibromo-5,5-dimethylhylhydantoin, sodium bromide, potassium bromide, and combinations thereof; and

B) optionally hydrogen peroxide.

Embodiment 50. The method of embodiment 44, wherein the inorganic base is selected from powder sodium hydroxide, sodium hydroxide solution, powder sodium acetate, and combinations thereof.

Embodiment 51. The method of embodiment 44, wherein the method step ii) of reacting the mixture occurs at a reaction temperature in the range of about 0° C. to about 70° C.

Embodiment 52. The method of embodiment 44, wherein the halogenation reagent is a mixture of about 1 part by weight hydrogen peroxide and about 2 parts by weight hydrogen bromide.

Embodiment 53. A method of preparing one or more compounds of Formula I, wherein

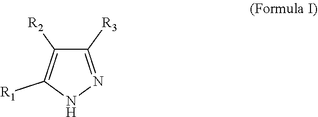

each of R₁, R₂, and R₃ is independently selected from halogen and hydrogen; and
wherein at least two of R₁, R₂, and R₃ are halogen, the method comprising

I) forming a mixture comprising
A) pyrazole or a pyrazole derivative;
B) a halogenation reagent;
C) water; and
D) optionally an inorganic base; and

II) reacting the mixture.

Embodiment 54. The method of embodiment 53, wherein the halogenation reagent comprises

A) a reagent selected from hydrogen bromide, bromine, N-bromosuccinimide, 1,3-dibromo-5,5-dimethylhylhydantoin, sodium bromide, potassium bromide, and combinations thereof; and

B) optionally hydrogen peroxide.

Embodiment 55. The method of embodiment 53, wherein the inorganic base is selected from powder sodium hydroxide, sodium hydroxide solution, powder sodium acetate, and combinations thereof.

Embodiment 56. The method of embodiment 53, wherein the method step of reacting the mixture occurs at a reaction temperature in the range of about 0° C. to about 70° C.

Embodiment 57. The method of embodiment 53, wherein the halogenation reagent is a mixture of about 1 part by weight hydrogen peroxide and about 2 parts by weight hydrogen bromide.

Embodiment 58. A method of preparing a compound of Formula VI, wherein

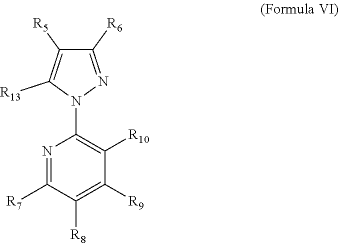

each of R₅-R₁₀ is independently selected from hydrogen and halogen; and
R₁₃ is an organic acid, the method comprising

I) forming a mixture comprising
A) a compound of Formula V, wherein

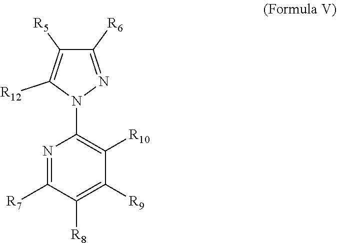

each of R₅-R₁₀ is independently selected from hydrogen and halogen;
R₁₂ is selected from ether, ester, and nitrile; and
wherein the compound of Formula V is prepared according to a method comprising
   i) forming a mixture comprising
      a) a compound of Formula III, wherein

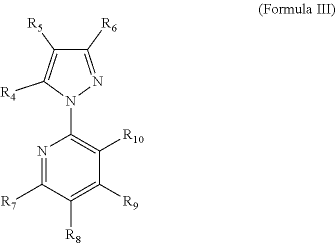

R₄ is hydrogen; and
each of R₅-R₁₀ is independently selected from hydrogen and halogen;
b) a solvent;
c) an organic compound;
d) a compound comprising a metal; and
e) optionally an additive; and
ii) reacting the mixture; and
B) a metal hydroxide; and

II) reacting the mixture and

III) adding an acid to the mixture.

Embodiment 59. The method of embodiment 58, wherein the metal hydroxide is selected from alkali hydroxide, alkaline earth metal hydroxide, and combinations thereof.

Embodiment 60. The method of embodiment 59, wherein the alkali hydroxide is selected from lithium hydroxide, sodium hydroxide, and potassium hydroxide.

Embodiment 61. The method of embodiment 59, wherein the alkaline earth metal hydroxide is selected from calcium hydroxide and barium hydroxide.

Embodiment 62. The method of embodiment 58, wherein the method step II) of reacting the mixture occurs at a reaction temperature in the range of about 0° C. to about 90° C.

Embodiment 63. The method of embodiment 58, wherein the compound comprising a metal is selected from a Grignard reagent and a lithium-containing compound.

Embodiment 64. The method of embodiment 63, wherein the Grignard reagent is selected from MeMgCl, iPrMgCl, iPrMgBr, EtMgCl, iPr₂NMgCl, iPr₂NMgBr, Et₂NMgCl, TMPMgCl, TMPMgCl.LiCl, iPr₂NMgCl.LiCl, iPr₂NMgBr.LiCl, and combinations thereof.

Embodiment 65. The method of embodiment 64, wherein the Grignard reagent is iPr₂NMgCl.B) optionally hydrogen peroxide.

Embodiment 66. The method of embodiment 63, wherein the lithium-containing compound is selected from LDA, nBuLi, and combinations thereof.

Embodiment 67. The method of embodiment 58, wherein the solvent is selected from THF, toluene, 1,4-dioxane, Me-THF, and combinations thereof.

Embodiment 68. The method of embodiment 67, wherein the solvent is THF.

Embodiment 69. The method of embodiment 58, wherein the organic compound is selected from dimethyl carbonate, N,N-dimethyacetamide, and combinations thereof.

Embodiment 70. The method of embodiment 69, wherein the organic compound is dimethyl carbonate.

Embodiment 71. The method of embodiment 58, wherein the method step ii) of reacting the mixture occurs at a reaction temperature in the range of about 0° C. to about 60° C.

Embodiment 72. The method of embodiment 71, wherein the method step ii) of reacting the mixture occurs at a reaction temperature in the range of about 0° C. to about 30° C.

Embodiment 73. The method of embodiment 58, wherein R₅ and R₆ of Formula III are each independently hydrogen.

Embodiment 74. The method of embodiment 58, wherein the compound of Formula III, is prepared according to a method comprising

I) forming a mixture comprising
A) a compound of Formula II, wherein

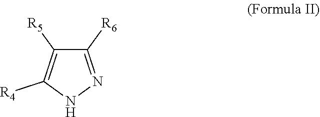

each of R₄, R₅, and R₆ is independently selected from hydrogen and halogen; and
wherein at least one of R₄, R₅, and R₆ is hydrogen;
B) a compound of Formula IV, wherein

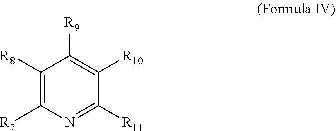

each of R₇-R₁₁ is independently selected from hydrogen and halogen;
C) a solvent;
D) an inorganic base; and
E) optionally an additive; and

II) reacting the mixture.

Embodiment 75. The method of embodiment 74, wherein the inorganic base is selected from powder sodium hydroxide, powder potassium hydroxide, potassium carbonate, potassium phosphate, powder sodium methoxide, powder potassium t-butoxide, and combinations thereof.

Embodiment 76. The method of embodiment 74, wherein the solvent is selected from toluene, N,N-dimethylformamide, N,N-dimethylacetamide, N-methyl-2-pyrrolidone, acetonitrile, and combinations thereof.

Embodiment 77. The method of embodiment 74, wherein the additive is selected from potassium iodide, a phase transfer catalyst, and combinations thereof.

Embodiment 78. The method of embodiment 77, wherein the phase transfer catalyst is selected from butyl ammonium chloride, tetra butyl ammonium bromide, aliquat-336, 18-crown-6, and combinations thereof.

Embodiment 79. The method of embodiment 74, wherein the method step of reacting the mixture occurs at a reaction temperature in the range of about 100° C. to about 200° C.

Embodiment 80. The method of embodiment 74, wherein the compound of Formula II is prepared according to a method comprising

I) forming a mixture comprising
A) one or more compounds of Formula I, wherein

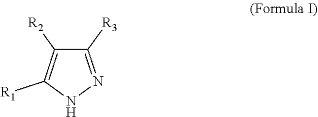

each of R₁, R₂, and R₃ is independently selected from halogen and hydrogen; and
wherein at least two of R₁, R₂, and R₃ are halogen;
B) optionally a dehalogenation reagent;
C) a reducing agent; and
D) a solvent; and

II) reacting the mixture.

Embodiment 81. The method of embodiment 80, wherein the solvent is selected from acetic acid, water, toluene, N,N-dimethylformamide, N,N-dimethylacetamide, and combinations thereof.

Embodiment 82. The method of embodiment 80, wherein the reducing agent is selected from sodium sulfite, sodium bisulfite, sodium hyposulfite, sodium thiosulfate, sodium hydrosulfide, sodium sulfate, and combinations thereof.

Embodiment 83. The method of embodiment 80, wherein the dehalogenation reagent is selected from sodium iodide, iodine, potassium iodide, tetra-n-butyl ammonium iodide, and combinations thereof.

Embodiment 84. The method of embodiment 80, wherein the method step of reacting the mixture occurs at a reaction temperature in the range of about 100° C. to about 180° C.

Embodiment 85. The method of embodiment 80, wherein the one or more compounds of Formula I are prepared according to a method comprising

I) forming a mixture comprising
A) pyrazole or a pyrazole derivative;
B) a halogenation reagent;
C) water; and
D) optionally an inorganic base; and

II) reacting the mixture.

Embodiment 86. The method of embodiment 85, wherein the halogenation reagent comprises

A) a reagent selected from hydrogen bromide, bromine, N-bromosuccinimide, 1,3-dibromo-5,5-dimethylhylhydantoin, sodium bromide, potassium bromide, and combinations thereof; and

B) optionally hydrogen peroxide.

Embodiment 87. The method of embodiment 85, wherein the inorganic base is selected from powder sodium hydroxide, sodium hydroxide solution, powder sodium acetate, and combinations thereof.

Embodiment 88. The method of embodiment 85, wherein the method step of reacting the mixture occurs at a reaction temperature in the range of about 0° C. to about 70° C.

Embodiment 89. The method of embodiment 85, wherein the halogenation reagent is a mixture of about 1 part by weight hydrogen peroxide and about 2 parts by weight hydrogen bromide.

Embodiment 90. A method of preparing a compound of Formula V, wherein

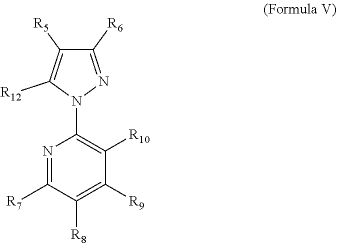

each of R₅-R₁₀ is independently selected from hydrogen and halogen;
R₁₂ is selected from ether, ester, and nitrile, the method comprising

I) forming a mixture comprising
A) a compound of Formula III, wherein

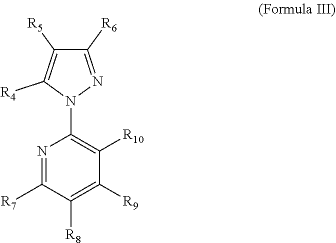

R₄ is hydrogen; and
each of R₅-R₁₀ is independently selected from hydrogen and halogen;
B) a solvent;
C) an organic compound;
D) a compound comprising a metal; and
E) optionally an additive; and

II) reacting the mixture.

Embodiment 91. The method of embodiment 90, wherein the compound comprising a metal is selected from a Grignard reagent and a lithium-containing compound.

Embodiment 92. The method of embodiment 91, wherein the Grignard reagent is selected from MeMgCl, iPrMgCl, iPrMgBr, EtMgCl, iPr₂NMgCl, iPr₂NMgBr, Et₂NMgCl, TMPMgCl, TMPMgCl.LiCl, iPr₂NMgCl.LiCl, iPr₂NMgBr.LiCl, and combinations thereof.

Embodiment 93. The method of embodiment 92, wherein the Grignard reagent is iPr₂NMgCl.

Embodiment 94. The method of embodiment 91, wherein the lithium-containing compound is selected from LDA, nBuLi, and combinations thereof.

Embodiment 95. The method of embodiment 90, wherein the solvent is selected from THF, toluene, 1,4-dioxane, Me-THF, and combinations thereof.

Embodiment 96. The method of embodiment 95, wherein the solvent is

THF.

Embodiment 97. The method of embodiment 90, wherein the organic compound is selected from dimethyl carbonate, N,N-dimethyacetamide, and combinations thereof.

Embodiment 98. The method of embodiment 97, wherein the organic compound is dimethyl carbonate.

Embodiment 99. The method of embodiment 90, wherein the method step II) of reacting the mixture occurs at a reaction temperature in the range of about 0° C. to about 60° C.

Embodiment 100. The method of embodiment 99, wherein the method step II) of reacting the mixture occurs at a reaction temperature in the range of about 0° C. to about 30° C.

Embodiment 101. The method of embodiment 90, wherein R₅ and R₆ of Formula III are each independently hydrogen.

Embodiment 102. The method of embodiment 90, wherein the compound of Formula III, is prepared according to a method comprising

I) forming a mixture comprising
A) a compound of Formula II, wherein

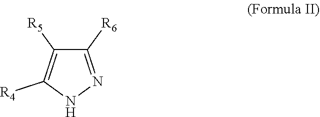

each of R₄, R₅, and R₆ is independently selected from hydrogen and halogen; and
wherein at least one of R₄, R₅, and R₆ is hydrogen;
B) a compound of Formula IV, wherein

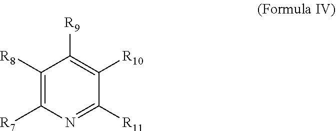

each of R₇-R₁₁ is independently selected from hydrogen and halogen;
C) a solvent;
D) an inorganic base; and
E) optionally an additive; and

II) reacting the mixture.

Embodiment 103. The method of embodiment 102, wherein the inorganic base is selected from powder sodium hydroxide, powder potassium hydroxide, potassium carbonate, potassium phosphate, powder sodium methoxide, powder potassium t-butoxide, and combinations thereof.

Embodiment 104. The method of embodiment 102, wherein the solvent is selected from toluene, N,N-dimethylformamide, N,N-dimethylacetamide, N-methyl-2-pyrrolidone, acetonitrile, and combinations thereof.

Embodiment 105. The method of embodiment 102, wherein the additive is selected from potassium iodide, a phase transfer catalyst, and combinations thereof.

Embodiment 106. The method of embodiment 105, wherein the phase transfer catalyst is selected from butyl ammonium chloride, tetra butyl ammonium bromide, aliquat-336, 18-crown-6, and combinations thereof.

Embodiment 107. The method of embodiment 102, wherein the method step of reacting the mixture occurs at a reaction temperature in the range of about 100° C. to about 200° C.

Embodiment 108. The method of embodiment 102, wherein the compound of Formula II is prepared according to a method comprising

I) forming a mixture comprising
A) one or more compounds of Formula I, wherein

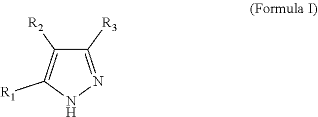

each of R₁, R₂, and R₃ is independently selected from halogen and hydrogen; and
wherein at least two of R₁, R₂, and R₃ are halogen;
B) optionally a dehalogenation reagent;
C) a reducing agent; and
D) a solvent; and

II) reacting the mixture.

Embodiment 109. The method of embodiment 108, wherein the solvent is selected from acetic acid, water, toluene, N,N-dimethylformamide, N,N-dimethylacetamide, and combinations thereof.

Embodiment 110. The method of embodiment 108, wherein the reducing agent is selected from sodium sulfite, sodium bisulfite, sodium hyposulfite, sodium thiosulfate, sodium hydrosulfide, sodium sulfate, and combinations thereof.

Embodiment 111. The method of embodiment 108, wherein the dehalogenation reagent is selected from sodium iodide, iodine, potassium iodide, tetra-n-butyl ammonium iodide, and combinations thereof.

Embodiment 112. The method of embodiment 108, wherein the method step of reacting the mixture occurs at a reaction temperature in the range of about 100° C. to about 180° C.

Embodiment 113. The method of embodiment 108, wherein the one or more compounds of Formula I are prepared according to a method comprising

I) forming a mixture comprising
A) pyrazole or a pyrazole derivative;
B) a halogenation reagent;
C) water; and
D) optionally an inorganic base; and

II) reacting the mixture.

Embodiment 114. The method of embodiment 113, wherein the halogenation reagent comprises

A) a reagent selected from hydrogen bromide, bromine, N-bromosuccinimide, 1,3-dibromo-5,5-dimethylhylhydantoin, sodium bromide, potassium bromide, and combinations thereof; and

B) optionally hydrogen peroxide.

Embodiment 115. The method of embodiment 113, wherein the inorganic base is selected from powder sodium hydroxide, sodium hydroxide solution, powder sodium acetate, and combinations thereof.

Embodiment 116. The method of embodiment 113, wherein the method step of reacting the mixture occurs at a reaction temperature in the range of about 0° C. to about 70° C.

Embodiment 117. The method of embodiment 113, wherein the halogenation reagent is a mixture of about 1 part by weight hydrogen peroxide and about 2 parts by weight hydrogen bromide.

Embodiment 118. A method of preparing a compound of Formula II-A, wherein

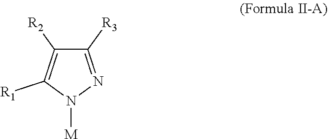

M is selected from alkali metals and alkaline metals;
each of R₄, R₅, and R₆ is independently selected from hydrogen and halogen; and
wherein at least one of R₄, R₅, and R₆ is hydrogen, the method comprising

I) forming a mixture comprising
A) one or more compounds of Formula I, wherein

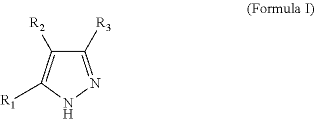

each of R₁, R₂, and R₃ is independently selected from halogen and hydrogen;
wherein at least two of R₁, R₂, and R₃ are halogen; and
wherein the one or more compounds of Formula I are prepared according to a method comprising
   i) forming a mixture comprising
      a) pyrazole or a pyrazole derivative;
      b) a halogenation reagent;
      c) water; and
      d) optionally an inorganic base; and
   ii) reacting the mixture.
B) optionally a dehalogenation reagent;
C) a reducing agent; and
D) a solvent; and

II) reacting the mixture.

Embodiment 119. The method of embodiment 118, wherein the solvent is selected from acetic acid, water, toluene, N,N-dimethylformamide, N,N-dimethylacetamide, and combinations thereof.

Embodiment 120. The method of embodiment 118, wherein the reducing agent is selected from sodium sulfite, sodium bisulfite, sodium hyposulfite, sodium thiosulfate, sodium hydrosulfide, sodium sulfate, and combinations thereof.

Embodiment 121. The method of embodiment 118, wherein the dehalogenation reagent is selected from sodium iodide, iodine, potassium iodide, tetra-n-butyl ammonium iodide, and combinations thereof.

Embodiment 122. The method of embodiment 118, wherein the method step II) of reacting the mixture occurs at a reaction temperature in the range of about 100° C. to about 180° C.

Embodiment 123. The method of embodiment 118, wherein the halogenation reagent comprises

A) a reagent selected from hydrogen bromide, bromine, N-bromosuccinimide, 1,3-dibromo-5,5-dimethylhylhydantoin, sodium bromide, potassium bromide, and combinations thereof; and

B) optionally hydrogen peroxide.

Embodiment 124. The method of embodiment 118, wherein the inorganic base is selected from powder sodium hydroxide, sodium hydroxide solution, powder sodium acetate, and combinations thereof.

Embodiment 125. The method of embodiment 118, wherein the method step ii) of reacting the mixture occurs at a reaction temperature in the range of about 0° C. to about 70° C.

Embodiment 126. The method of embodiment 118, wherein the halogenation reagent is a mixture of about 1 part by weight hydrogen peroxide and about 2 parts by weight hydrogen bromide.

Embodiment 127. The method of embodiment 118, wherein M is selected from lithium, sodium, potassium, calcium, and magnesium.

Embodiment 128. The method of embodiment 118, wherein the compound of Formula II-A is

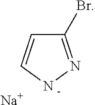

Embodiment 129. A compound of Formula II-A, wherein

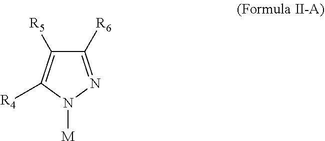

M is selected from alkali metals and alkaline metals;
each of R₄, R₅, and R₆ is independently selected from hydrogen and halogen; and
wherein at least one of R₄, R₅, and R₆ is hydrogen.

Embodiment 130. The compound of embodiment 129, wherein the compound is

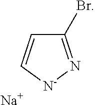

In one aspect, a compound of Formula VI is prepared according to a method represented by Scheme 1. The R groups are as defined anywhere in this disclosure.

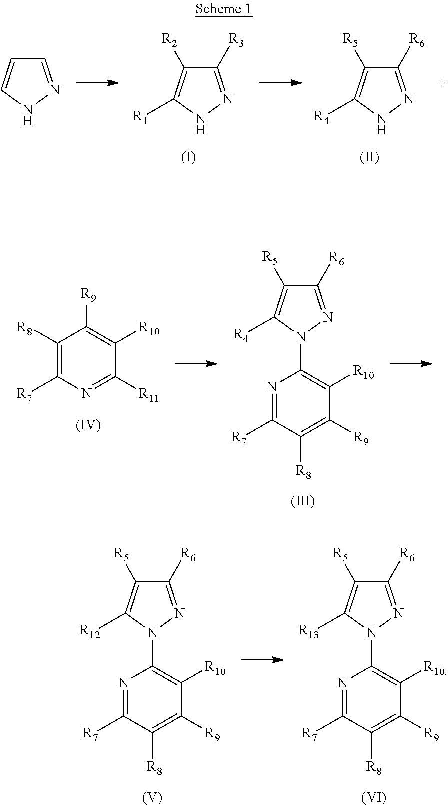

In one aspect, a compound of Formula VI is prepared according to a method represented by Scheme 2. The R groups are as defined anywhere in this disclosure. Formula IA and Formula IB represent two distinct compounds of Formula I.

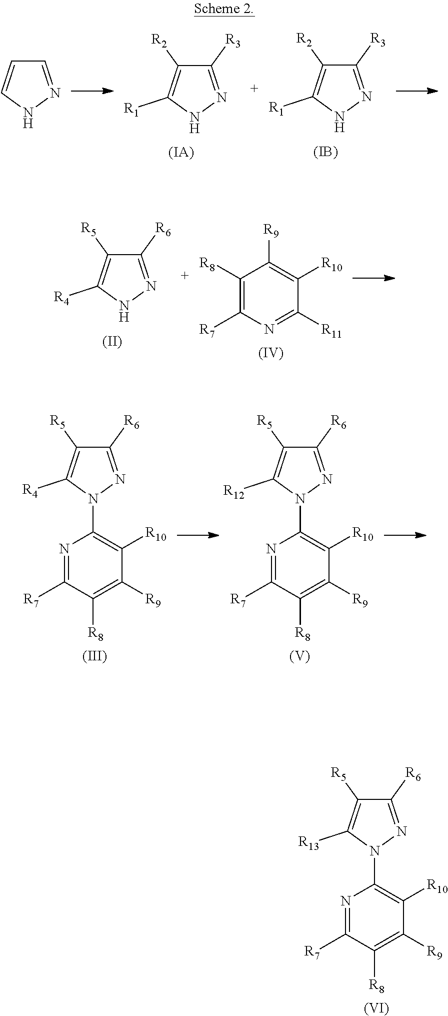

In one aspect, a compound of Formula VI is prepared according to a method represented by Scheme 3. The R groups are as defined anywhere in this disclosure.

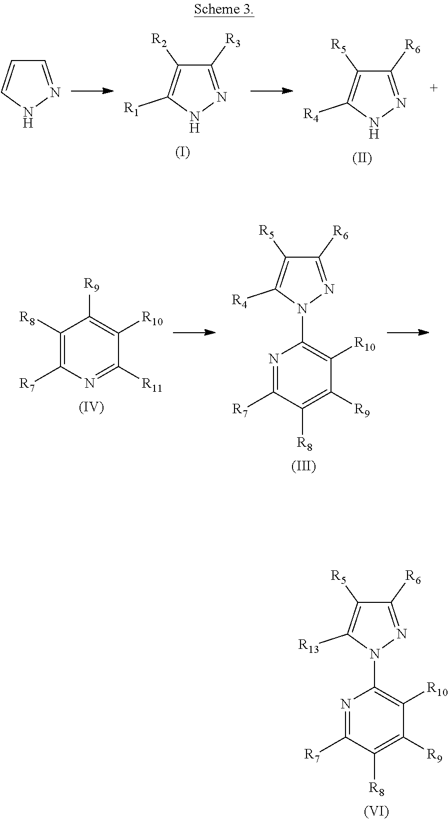

In one aspect, a compound of Formula VI is prepared according to a method represented by Scheme 4. The R groups are as defined anywhere in this disclosure. Formula IA and Formula IB represent two distinct compounds of Formula I.

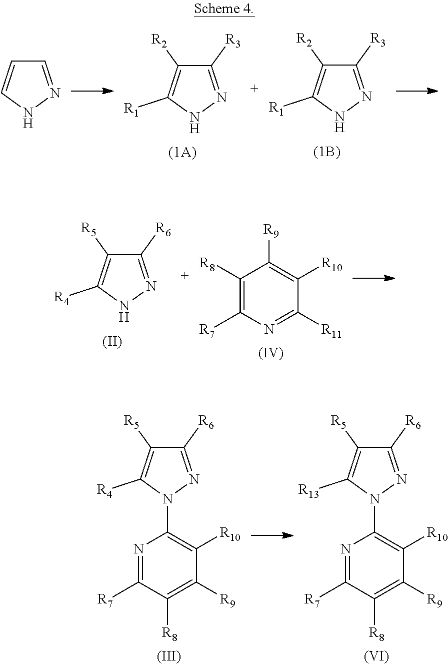

In one aspect, 5-Bromo-2-(3-chloro-pyridin-2-yl)-2H-pyrazole-3-carboxylic acid is prepared according to a method represented by Scheme 5.

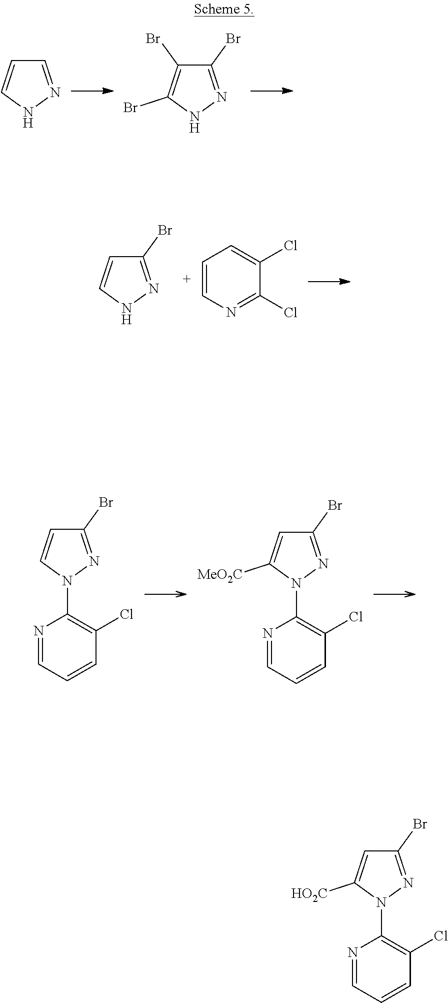

In one aspect, 5-Bromo-2-(3-chloro-pyridin-2-yl)-2H-pyrazole-3-carboxylic acid is prepared according to a method represented by Scheme 6.

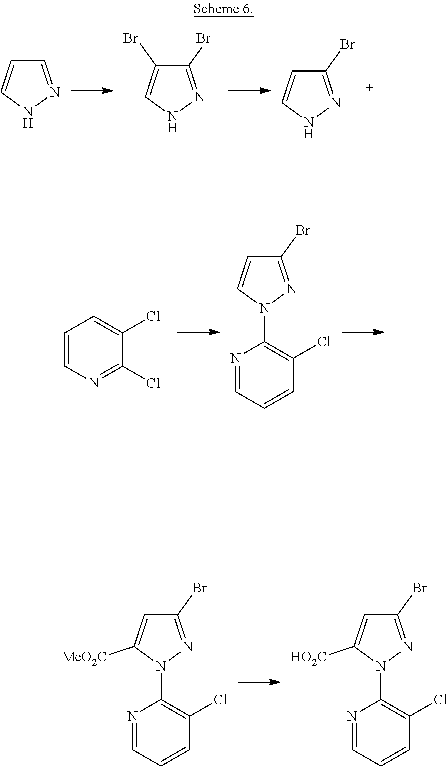

In one aspect, 5-Bromo-2-(3-chloro-pyridin-2-yl)-2H-pyrazole-3-carboxylic acid is prepared according to a method represented by Scheme 7.

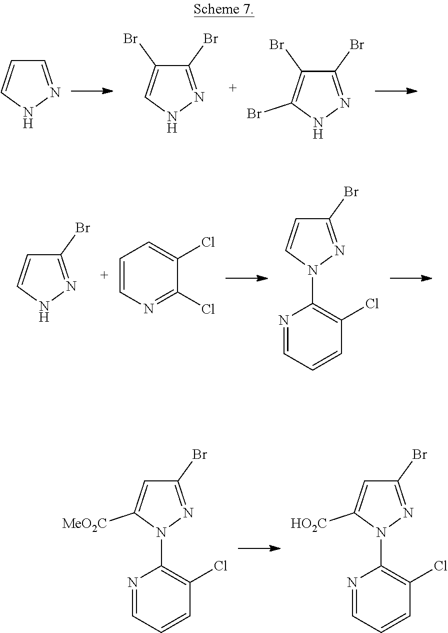

In one aspect, 5-Bromo-2-(3-chloro-pyridin-2-yl)-2H-pyrazole-3-carboxylic acid is prepared according to a method represented by Scheme 8.

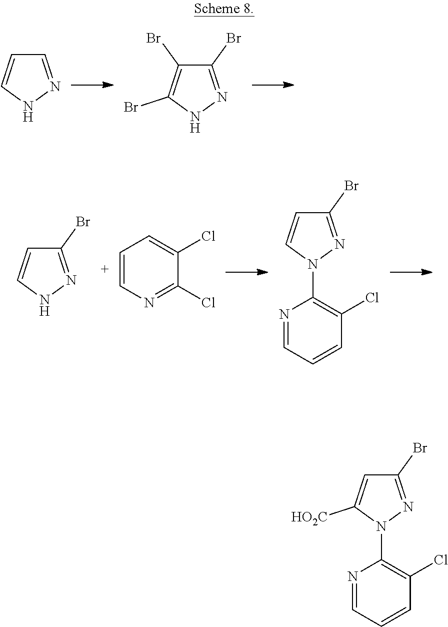 [SMILES: c1cn[nH]c1 ; Brc1n[nH]c(Br)c1Br ; Brc1cc[nH]n1 ; Clc1cccnc1Cl ; Clc1cccnc1-n1ccc(Br)n1 ; O=C(O)c1cc(Br)nn1-c1ncccc1Cl]

In one aspect, 5-Bromo-2-(3-chloro-pyridin-2-yl)-2H-pyrazole-3-carboxylic acid is prepared according to a method represented by Scheme 9.

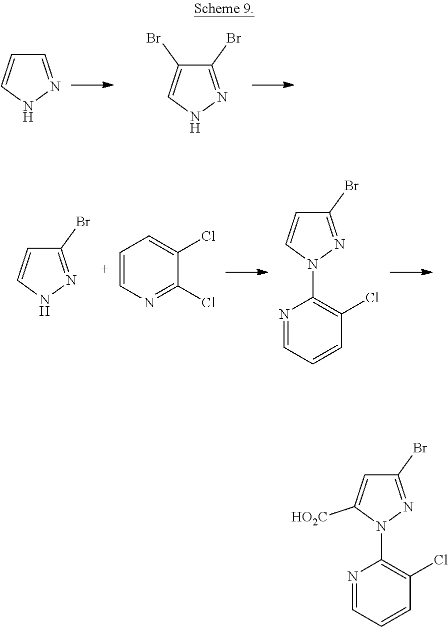 [SMILES: c1cn[nH]c1 ; Brc1c[nH]nc1Br ; Brc1cc[nH]n1 ; Clc1cccnc1Cl ; Clc1cccnc1-n1ccc(Br)n1 ; O=C(O)c1cc(Br)nn1-c1ncccc1Cl]

In one aspect, 5-Bromo-2-(3-chloro-pyridin-2-yl)-2H-pyrazole-3-carboxylic acid is prepared according to a method represented by Scheme 10.

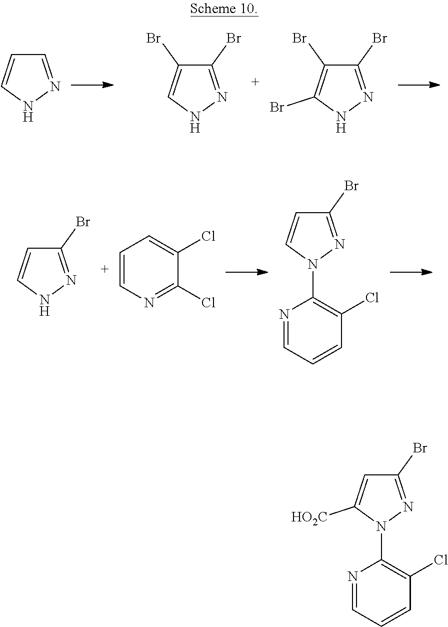

In one aspect, at least one distinct compound of Formula I is prepared according to a method represented by Scheme 11. The R groups are as defined anywhere in this disclosure.

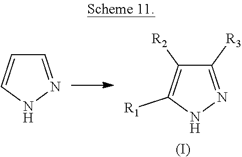

This aspect includes reacting pyrazole with a halogenation reagent in water and optionally in the presence of an inorganic base. In one embodiment, the halogenation reagent is selected from hydrogen peroxide/HBr, bromine (Br₂), N-bromosuccinimide (NBS), 1,3-dibromo-5,5-dimethylhylhydantoin, hydrogen peroxide/NaBr, hydrogen peroxide/KBr, hydrogen peroxide/Br₂, and combinations thereof. In another embodiment, the halogenation reagent is hydrogen peroxide/HBr. In one embodiment, the inorganic base is selected from powder sodium hydroxide, sodium hydroxide solution, powder sodium acetate, and combinations thereof. In another embodiment, there is no base. In one embodiment, the reaction temperature is in the range from about 0° C. to about 40° C. In another embodiment, the reaction temperature is in the range from about 0° C. to 20° C. The relative amounts of distinct compounds of Formula I produced by the reaction can be controlled according to the ratio of hydrogen peroxide/HBr. In one embodiment, hydrogen bromide and hydrogen peroxide and pyrazole are present in a ratio in the range from about 2 eq: 2 eq: 1 eq to about 10 eq: 2 eq: 1 eq. In another embodiment, hydrogen bromide and hydrogen peroxide and pyrazole are present in a ratio of 4 eq: 2 eq: 1 eq. In one embodiment, the halogenation reagent is a mixture of about 1 part by weight hydrogen peroxide and about 1 part by weight hydrogen bromide. In another embodiment, the halogenation reagent is a mixture of about 1 part by weight hydrogen peroxide and about 2 parts by weight hydrogen bromide. In another embodiment, the halogenation reagent is a mixture of about 1 part by weight hydrogen peroxide and about 5 parts by weight hydrogen bromide

In one aspect, a compound of Formula II is prepared according to a method represented by Scheme 12. The R groups are as defined anywhere in this disclosure.

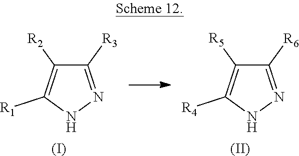

This aspect includes reacting at least one distinct compound of Formula I with a dehalogenation reagent in a solvent in the presence of a reducing agent. In one embodiment, the solvent is selected from water, acetic acid, toluene, N,N-Dimethylformamide (DMF), N,N-Dimethylacetamide(DMAc), and combinations thereof. In a preferred embodiment, the solvent is water. In another embodiment, the dehalogenation reagent is selected from sodium iodide, iodine, potassium iodide, tetra-n-butyl ammonium iodide (TBAI), and combinations thereof. In another embodiment, the dehalogenation reagent is potassium iodide. In one embodiment, the reducing agent is selected from sodium sulfite, sodium bisulfite, sodium hyposulfite, sodium thiosulfate, sodium hydrosulfide, sodium sulphate, and combinations thereof. In another embodiment, the reducing agent is sodium sulfite. In one embodiment, the reaction temperature is in the range from about 100° C. to about 180° C. In another preferred embodiment, the reaction temperature ranges from about 160° C. to about 180° C.

In one aspect, a compound of Formula II-A is prepared according to a method represented by Scheme 13. The R groups are as defined anywhere in this disclosure.

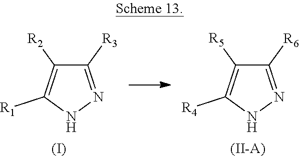

In one embodiment, the compound of Formula II-A is a metal salt of Formula II. In one embodiment, the bond between M and N is an ionic bond. In one embodiment, M is selected from alkali metals and alkaline metals. In one embodiment, M is selected from lithium, sodium, and potassium. In another embodiment, M is sodium. In another embodiment, M is selected from calcium and magnesium.

In one embodiment, the compound of Formula II-A is

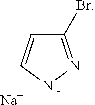

This aspect includes reacting at least one distinct compound of Formula I with a dehalogenation reagent in a solvent in the presence of a reducing agent. In one embodiment, the solvent is selected from water, acetic acid, toluene, N,N-Dimethylformamide (DMF), N,N-Dimethylacetamide(DMAc), and combinations thereof. In a preferred embodiment, the solvent is water. In another embodiment, the dehalogenation reagent is selected from sodium iodide, iodine, potassium iodide, tetra-n-butyl ammonium iodide (TBAI), and combinations thereof. In another embodiment, the dehalogenation reagent is potassium iodide. In one embodiment, the reducing agent is selected from sodium sulfite, sodium bisulfite, sodium hyposulfite, sodium thiosulfate, sodium hydrosulfide, sodium sulphate, and combinations thereof. In another embodiment, the reducing agent is sodium sulfite. In one embodiment, the reaction temperature is in the range from about 100° C. to about 180° C. In another preferred embodiment, the reaction temperature ranges from about 160° C. to about 180° C.

In one aspect, a compound of Formula III is prepared according to a method represented by Scheme 14. The R groups are as defined anywhere in this disclosure.

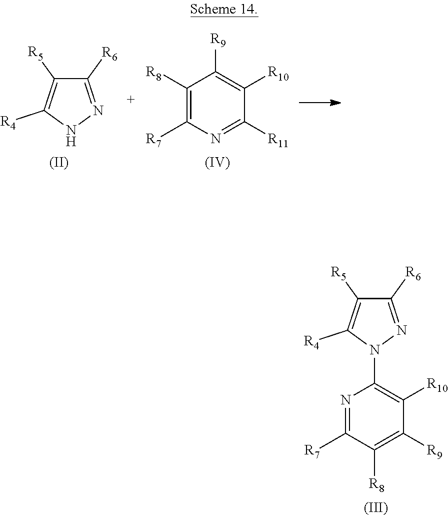

This aspect includes mixing a compound of Formula II with a compound of Formula IV in a solvent in the presence of an inorganic base and optionally an additive. In one embodiment, the inorganic base is selected from powder sodium hydroxide, powder potassium hydroxide, potassium carbonate, potassium phosphate, powder sodium methoxide, powder potassium t-butoxide, and combinations thereof. In one embodiment, the solvent is selected from toluene, N,N-Dimethylformamide (DMF), N,N-Dimethylacetamide (DMAc) and N-methyl-2-pyrrolidone (NMP), acetonitrile, and combinations thereof. In another embodiment, the solvent is toluene. In one embodiment, the reaction temperature is in the range from about 100° C. to about 200° C. In another embodiment, the reaction temperature is in the range from about 130° C. to about 180° C. In another embodiment, the temperature is 145° C. to about 160° C.

In one aspect, a compound of Formula VI is prepared according to a method represented by Scheme 15. The R groups are as defined anywhere in this disclosure.

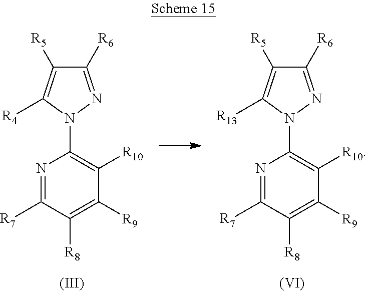

This aspect includes mixing a compound of Formula III with CO₂ in a solvent in the presence of a base reagent and optionally an additive. In one embodiment, the base reagent is selected from MeMgCl, iPrMgCl, iPrMgBr, EtMgCl, LDA, nBuLi, iPr₂NMgCl, iPr₂NMgBr, Et₂NMgCl, TMPMgCl, TMPMgCl.LiCl, iPr₂NMgCl.LiCl, iPr₂NMgBr.LiCl, and combinations thereof. In another embodiment, the base reagent is iPr₂NMgCl. In one embodiment, the solvent is selected from THF, toluene, 1,4-dioxane, Me-THF, and combinations thereof. In another embodiment, the solvent is THF. In one embodiment, the reaction temperature is in the range from about 0° C. to about 60° C. In another embodiment, the temperature is in the range from about 0° C. to about 30° C.

In one aspect, a compound of Formula V is prepared according to a method represented by Scheme 16. The R groups are as defined anywhere in this disclosure.

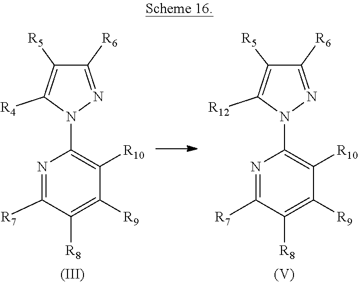

This aspect includes mixing a compound of Formula III with dimethyl carbonate (DMC) in a solvent in the presence of a base reagent and optionally an additive. In one embodiment, the base reagent is selected from MeMgCl, iPrMgCl, iPrMgBr, EtMgCl, LDA, nBuLi, iPr₂NMgCl, iPr₂NMgBr, Et₂NMgCl, TMPMgCl, TMPMgCl.LiCl, iPr₂NMgCl.LiCl, iPr₂NMgBr.LiCl, and combinations thereof. In another embodiment, the base reagent is iPr₂NMgCl. In one embodiment, the solvent is selected from THF, toluene, 1,4-dioxane, 2-Me-THF, and combinations thereof. In another embodiment, the solvent is THF. In one embodiment, the reaction temperature is in the range from about 0° C. to about 60° C. In another embodiment, the temperature is in the range from about 0° C. to about 30° C.

In one aspect, a compound of Formula VI is prepared according to a method represented by Scheme 17. The R groups are as defined anywhere in this disclosure.

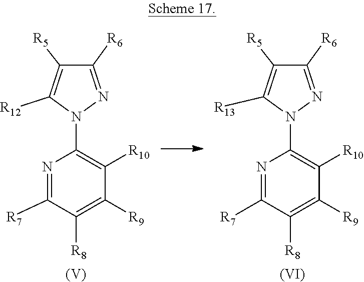

This aspect includes reacting a compound of Formula V with an aqueous metal hydroxide solution and adding an acid. In one embodiment, the metal hydroxide is selected from alkali hydroxide, alkaline earth metal hydroxide, and combinations thereof. In one embodiment, the alkali hydroxide is selected from lithium hydroxide, sodium hydroxide, potassium hydroxide, and combinations. In one embodiment, the alkaline earth metal hydroxide is selected from calcium hydroxide, barium hydroxide, and combinations thereof. In another embodiment, the metal hydroxide is sodium hydroxide or potassium hydroxide. In one embodiment, the reaction temperature is in the range from about 0° C. to about 90° C. In another embodiment, the reaction temperature is in the range from about 60° C. to about 80° C. In another embodiment, the reaction temperature is in the range from about 40° C. to about 70° C. In one embodiment, the acid is selected from dilute sulfuric acid, dilute hydrochloric acid, and dilute phosphoric acid. In another embodiment, the acid is dilute sulfuric acid.

### EXAMPLES

Without further elaboration, it is believed that one skilled in the art using the preceding description can utilize the present invention to its fullest extent. The following Examples are, therefore, to be construed as merely illustrative, and not limiting of the disclosure in any way whatsoever. The starting material for the following Examples may not have necessarily been prepared by a particular preparative run whose procedure is described in other Examples. It also is understood that any numerical range recited herein includes all values from the lower value to the upper value. For example, if a range is stated as 10-50, it is intended that values such as 12-30, 20-40, or 30-50, etc., are expressly enumerated in this specification. These are only examples of what is specifically intended, and all possible combinations of numerical values between and including the lowest value and the highest value enumerated are to be considered to be expressly stated in this application.

### Example 1. Hydrogen Peroxide/HBr as a Halogenation Reagent

34 grams of pyrazole was dissolved in 100 g water and charged to a reactor with 337.2 g of 48% hydrogen bromide solution. 170 g of 30% hydrogen peroxide was added drop-wise at 0° C. over 2 hours. The reaction temperature was controlled at 0-20° C. After reaction, the product was precipitated as a solid, and then the reaction mixture was quenched with 10% sodium sulfite. After filtration and drying, 142 g of high purity (97%, LC Area) of a mixture of 3,4,-dibromo-1H-pyrazole and 3,4,5-tribromo-1H-pyrazole is obtained (the ratio of 3,4-dibromo-1H-pyrazole to 3,4,5-tribromo-1H-pyrazole =9:1, LC Area).

### Example 2. Bromine/Sodium Hydroxide as a Halogenation Reagent

34 grams of pyrazole was dissolved in water and then sodium hydroxide was added at 0° C. to obtain the corresponding pyrazole sodium salt. Next, 239.7 g of bromine was added drop-wise at 0° C. over 2 hours. The reaction temperature was controlled at 20-40° C. After reaction, the product was precipitated as a solid, and then the reaction mixture was quenched with 10% sodium sulfite. After filtration and drying, 147 g of high purity (98%, LC Area) of 3,4,5-tribromo-1H-pyrazole was obtained.

### Example 3. Potassium Iodide/Sodium Sulfite as a Dehalogenation Reagent

100 grams of a mixture of 3,4,5-tribromo-1H-pyrazole and 3,4,-dibromo-1H-pyrazole (the ratio of 3,4-dibromo-1H-pyrazole to 3,4,5-tribromo-1H-pyrazole =9:1, LC Area), 111 g of Na₂SO₃ in 500 mL water were reacted at 160-180° C. for 4 hours to complete reaction. After completion of the reaction, the reaction mixture was extracted with methyl isobutyl ketone (MIBK), and concentrated under vacuum at 40-45° C. to obtain 3-bromo-1H-pyrazole as a solid.

### Example 4. Coupling Reaction

11.5 grams of 3-bromo-1H-pyrazole and 14.7 g of 40% NaOH solution were reacted in the presence of toluene at a temperature of 150-160° C. Water was removed by azetropic distillation under reflux temperature to yield the corresponding 3-bromo-1H-pyrazole sodium salt. Then, toluene and 13.5 g of 2,3-dichloropyridine were added and the mixture was reacted at 150-160° C. After reaction, the reaction mixture was quenched with water and the organic layer was separated. The organic layer was then washed with water and concentrated under vacuum at 40-45° C. to obtain 2-(3-bromo-1H-pyrazol-1-yl)-3-chloropyridine as a solid.

### Example 5. Reaction in the Presence of a Grignard Reagent

32 grams of 2-(3-bromo-1H-pyrazol-1-yl)-3-chloropyridine was dissolved in THF then iPr₂NMgCl (in situ MeMgCl and iPr₂NH) was added at 0° C. to yield the corresponding 2-(3-bromo-1H-pyrazol-1-yl)-3-chloropyridine magnesium salt. After 2.5 hours, 52.0 g of DMC was added drop-wise at room temperature over 6 hours. The reaction temperature was controlled at 20-40° C. After reaction, THF and DMC were distilled off under reduced pressure, and then the reaction mixture was quenched with water. Next, toluene was added. After separation and concentration, 36.7 g of high purity (90%, LC Area) of methyl 3-bromo-1-(3-chloropyridin-2-yl)-1H-pyrazole-5-carboxylate was obtained.

### Example 6. Hydrolysis

30 grams of methyl 3-bromo-1-(3 -chloropyridin-2-yl)-1H-pyrazole-5-carboxylate and a caustic soda solution in 90 g of toluene were charged to a flask at 85° C. The the mixture was kept at 80-85° C. for 2 hours to complete reaction. The aqueous phase was washed with toluene twice. Dilute H₂SO₄ was used to adjust pH to about 1. After filtration and drying, 26.5 g (97%, LC Area) of 5-bromo-2-(3-chloro-pyridin-2-yl)-2H-pyrazole-3-carboxylic acid was obtained.

### Example 7. Carboxylation

32 grams of 2-(3-bromo-1H-pyrazol-1-yl)-3-chloropyridine was dissolved in THF and then iPr₂NMgCl, obtained in situ by separately adding MeMgCl and iPr2NH, was added at 0° C. to yield the corresponding 2-(3-bromo-1H-pyrazol-1-yl)-3-chloropyridine magnesium salt. After 2.5 hours, CO2 gas was added at room temperature over 1 hour. The reaction temperature was controlled at 20-40° C. After reaction, THF was distilled off under reduced pressure, and then the reaction mixture was quenched with water. Next, toluene was added. After separation and concentration, 30.0 g of high purity (95%, LC Area) of 5-bromo-2-(3-chloro-pyridin-2-yl)-2H-pyrazole-3-carboxylic acid was obtained.

This written description uses examples to illustrate the present disclosure, including the best mode, and also to enable any person skilled in the art to practice the disclosure, including making and using any devices or systems and performing any incorporated methods. The patentable scope of the disclosure is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A method of preparing a compound of Formula VI, wherein 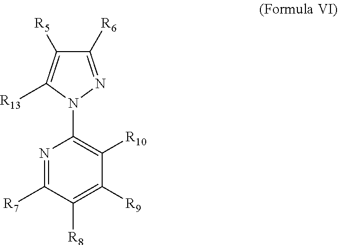
each of R₅-R₁₀ is independently selected from hydrogen and halogen; and
R₁₃ is an organic acid, the method comprising
I) forming a mixture comprising
A) a compound of Formula III, wherein 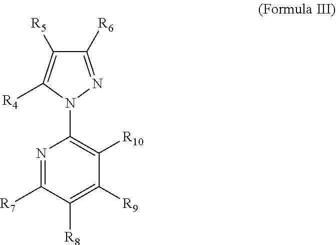
R₄ is hydrogen; and
each of R₅-R₁₀ is independently selected from hydrogen and halogen;
B) a carbonyl-containing compound;
C) a solvent;
D) a compound comprising a metal; and
E) optionally an additive; and
II) reacting the mixture.

2. The method of claim 1, wherein the compound comprising a metal is selected from a Grignard reagent and a lithium-containing compound.

3. The method of claim 2, wherein the Grignard reagent is selected from MeMgCl, iPrMgCl, iPrMgBr, EtMgCl, iPr₂NMgCl, iPr₂NMgBr, Et₂NMgCl, TMPMgCl, TMPMgCl.LiCl, iPr₂NMgCl.LiCl, iPr₂NMgBr.LiCl, and combinations thereof.

4. The method of claim 3, wherein the Grignard reagent is iPr₂NMgCl.

5. The method of claim 2, wherein the lithium-containing compound is selected from LDA, nBuLi, and combinations thereof.

6. The method of claim 1, wherein the solvent is selected from THF, toluene, 1,4-dioxane, Me-THF, and combinations thereof.

7. The method of claim 6, wherein the solvent is THF.

8. The method of claim 1, wherein the carbonyl-containing compound is selected from dimethylcarbonate, N,N-dimethylacetamide, carbon dioxide, and combinations thereof.

9. The method of claim 8, wherein the carbonyl-containing compound is carbon dioxide.

10. The method of claim 1, wherein the compound of Formula III, is prepared according to a method comprising
I) forming a mixture comprising
A) a compound of Formula II, wherein 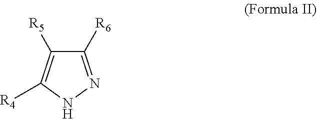
each of R₄, R₅, and R₆ is independently selected from hydrogen and halogen; and
wherein at least one of R₄, R₅, and R₆ is hydrogen;
B) a compound of Formula IV, wherein 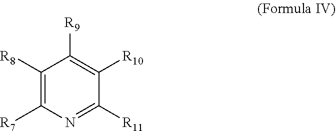
each of R₇-R₁₁ is independently selected from hydrogen and halogen;
C) a solvent;
D) an inorganic base; and
E) optionally an additive; and
II) reacting the mixture.

11. The method of claim 10, wherein the inorganic base is selected from powder sodium hydroxide, powder potassium hydroxide, potassium carbonate, potassium phosphate, powder sodium methoxide, powder potassium t-butoxide, and combinations thereof.

12. The method of claim 10, wherein the solvent is selected from toluene, N,N-dimethylformamide, N,N-dimethylacetamide, N-methyl-2-pyrrolidone, acetonitrile, and combinations thereof.

13. The method of claim 10, wherein the additive is selected from potassium iodide, a phase transfer catalyst, and combinations thereof.

14. The method of claim 13, wherein the phase transfer catalyst is selected from butyl ammonium chloride, tetra butyl ammonium bromide, aliquat-336, 18-crown-6, and combinations thereof.

15. A method of preparing a compound of Formula III, wherein 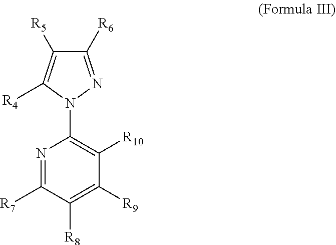
R₄ is hydrogen; and
each of R₅-R₁₀ is independently selected from hydrogen and halogen, the method comprising
I) forming a mixture comprising
A) a compound of Formula II, wherein 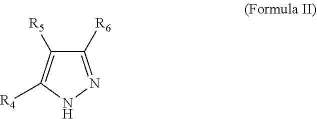
each of R₄, R₅, and R₆ is independently selected from hydrogen and halogen;
wherein at least one of R₄, R₅, and R₆ is hydrogen; and
wherein the compound of Formula II is prepared according to a method comprising
i) forming a mixture comprising
a) one or more compounds of Formula I, wherein 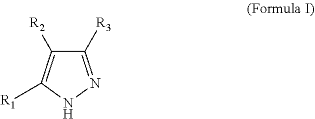
each of R₁, R₂, and R₃ is independently selected from halogen and hydrogen; and
wherein at least two of R₁, R₂, and R₃ are halogen;
b) optionally a dehalogenation reagent;
c) a reducing agent; and
d) a solvent; and
ii) reacting the mixture.
B) a compound of Formula IV, wherein 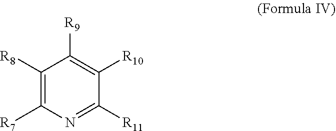
each of R₇R₁₁ is independently selected from hydrogen and halogen;
C) a solvent;
D) an inorganic base; and
E) optionally an additive; and
II) reacting the mixture.

16. The method of claim 15, wherein the inorganic base is selected from powder sodium hydroxide, powder potassium hydroxide, potassium carbonate, potassium phosphate, powder sodium methoxide, powder potassium t-butoxide, and combinations thereof.

17. The method of claim 15, wherein the solvent C) is selected from toluene, N,N-dimethylformamide, N,N-dimethylacetamide, N-methyl-2-pyrrolidone, acetonitrile, and combinations thereof.

18. The method of claim 15, wherein the additive is selected from potassium iodide, a phase transfer catalyst, and combinations thereof.

19. A compound of Formula II-A, wherein 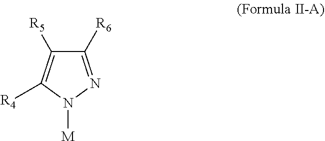
M is selected from alkali metals and alkaline metals;
each of R₄, R₅, and R₆ is independently selected from hydrogen and halogen; and
wherein at least one of R₄, R₅, and R₆ is hydrogen.

20. The compound of claim 19, wherein the compound is [chemical structure]
